(19) United States
(12) Patent Application Publication
(10) Pub. No.: US 2012/0295321 A1
(43) Pub. Date: Nov. 22, 2012

(54) Pentose Transporters and Uses Thereof

(75) Inventors: Patricia Ann vanKuyk, Utrecht (NL); Ronald Peter de Vries, Utrecht (NL); Jacob Visser, Wageningen (NL); Arthur Franciscus Johannes Ram, Hillegom (NL); Cornelis Antonius Maria Jacobus Johannes van den Hondel, Gouda (NL); Evy Battaglia, Utrecht (NL); Diana Alexandra Vivas Duarte, Utrecht (NL)

(73) Assignees: Universiteit Utrecht Holding B.V., Utrecht (NL); Universiteit Leiden, Leiden (NL); Stichting voor de Technische Wetenschappen, Utrecht (NL); Vereniging voor christelijk hoger onderwijs, wetenschappelijk onderzoek en patientënzorg, Amsterdam (NL)

(21) Appl. No.: 13/509,299

(22) PCT Filed: Nov. 12, 2010
(86) PCT No.: PCT/NL2010/050755

§ 371 Date: Jul. 27, 2012

(30) Foreign Application Priority Data
Nov. 12, 2009 (NL) PCT/NL2009/050683

(51) Int. Cl. C12P 7/10; C12N 1/19; C12N 1/15

(52) U.S. Cl. 435/165; 435/254.21; 435/254.3; 435/254.6; 435/254.11; 435/254.2

(57) ABSTRACT

The invention relates to the production of biofuels, proteins, peptides and other value-added compounds from crude carbon sources. The inventors identified genes encoding novel pentose transporters, in particular transporters of L-arabinose and/or D-xylose. Regulation of the Aspergillus niger genes by xlnR and araR was instrumental in the identification of these genes and their substrate specificities. Provided are novel pentose transporters and their encoding nucleic acids. Also provided are host cells (over)expressing a transporter, and industrial applications thereof, for instance in biofuel production.

The invention relates to the production of biofuels and other value-added compounds from crude carbon sources. In particular, it relates to methods for converting at least part of a lignocellulosic crude carbon source into a value-added compound by a host cell, and to host cells for use in such methods.

The utilization of crude carbon source (mainly plant biomass) is receiving an increasing interest from the industry, not only with respect to established fermentations but also to novel products such as bio-ethanol. Ethanol production from renewable material is a sustainable alternative to the use of fossil fuels. Bioethanol for transportation fuel can be produced in a sustainable way by fermentation of lignocellulosic raw materials, such as agricultural and forestry waste or energy crops. Other value added compounds include proteins, like enzymes, and peptides. For example the molasses left over from sugar production from sugar beets can be used for the production of proteins and peptides. See Siqueira et al. (2008) Bioresour. Technol. 99(17): 8156-63, Alriksson et al. (2009) Appl. Environ. Microbiol. 75(8):2366-74, Peixoto-Nogueira Sde C et al. (2009) J. Ind. Microbiol. Technol 36(1):149-55. He et al. disclose ergosterol production from molasses by genetically modified Saccharomyces cerevisiae. (2007, Appl. Microbiol. Biotechnol. 75:55-60); Ghazi et al. describe beet syrup and molasses as low-cost feedstock for the enzymatic production of fructo-oligosaccharides. (2006, J. Agric. Food Chem. 54(8):2964-8).

For the choice of the fermenting microorganism, complete substrate utilization, inhibitor tolerance and ethanol productivity are important aspects. There are strains of the yeast S. cerevisiae known which satisfy the last two conditions. However, metabolic engineering is required to obtain strains able to ferment e.g. L-arabinose and D-xylose, the most abundant pentose sugars in hemicellulose. Although present in a smaller fraction than D-xylose, also L-arabinose needs to be efficiently converted to ethanol for overall process economy. Furthermore, L-arabinose conversion to ethanol reduces carbon sources to be used by contaminant organisms competing with yeast.

Thus, sustainable production of biofuel ethanol from e.g. wheat straw, corn stover, bagasse and wood hydrolysates requires the fermentation of both the hexose and the pentose fractions. Efforts are being made to ferment lignocellulose hydrolysates to ethanol. Although modified S. cerevisiea strains have been designed which are capable of growth on and fermentation of D-xylose, their growth rate is still poor. Previously, xylose utilisation has been achieved by the heterologous expression of NAD(P)H-dependent xylose reductase (XR) and NAD+-dependent xylitol dehydrogenase (XDH) from Pichia stipitis. However, the cofactor imbalance between the two enzymes generated low ethanol yield and productivity. Attempts to express bacterial xylose isomerase (XI) genes have also given limited results due to a low enzyme expression and to the inhibition of XI by xylitol. Xylose utilisation can also be limited by transport, by a low xylulokinase level and low level of the pentose phosphate pathway.

In view of the current emphasis on pentose to ethanol fermentations for e.g. biofuel production, there is an urgent need for the development of improved host yeast strains. In addition, an enhanced utilization of pentoses by yeast and other industrial filamentous fungi such as Aspergillus or Trichoderma would allow for improved fermentations on crude carbon substrates, thereby widening the possible applications of host cells displaying modified pentose transport. In the case of host cell co-cultivation, for example on D-glucose and D-xylose, normally one sugar is used preferentially. For example, D-glucose is used first after which D-xylose is metabolised. This often results in a biphasic growth curve, due to different growth rates for the different sugars. A strain which expresses heterologous transporters (such as pentose transporters), or homologous transporters under control of other promoters (for example a constitutive promoter), could result in a cell in which two (or more) sugars (such as D-glucose and D-xylose) are utilized simultaneously allowing for improved growth.

The present inventors therefore set out to identify genes encoding novel pentose transporters, in particular transporters of L-arabinose or D-xylose. It was found that regulation of the Aspergillus niger genes by xlnR (xylose responsive positively acting regulator) and araR (the L-arabinose responsive positively acting regulator) was instrumental in the identification of these genes and their substrate specificities. This was determined by comparing micro-array data from regulator deletion strains to the reference strain transferred to the relevant pentose sugars. Genes with a greater than 4.5-fold change in transcript level were selected for further biochemical analysis. This led to the identification of 8 novel polypeptide sequences and their encoding nucleic acids. More in particular, they identified the transporter proteins An08g01720 (herein also abbreviated as 1720), An03g01620 (1620), An11g01100) (1100), An06g00560 (0560), An02g08230 (8230), An07g00780 (0780), An13g02590 (2590) and An03g02190 (2190). The proteins are encoded by, respectively, the genes An08g01720, An03g01620, An11g01100, An06g00560, An02g08230, An07g00780, An13g02590 and An03g02190. The protein and cDNA sequences of the novel transporters can be found in FIGS. 1-8.

The invention therefore relates to a polypeptide selected from the group consisting of: a) a polypeptide having an amino acid sequence showing at least 80% identity with an amino acid sequence shown in FIG. 1A, 2A, 3A, 4A, 5A, 6A, 7A or 8A and showing in vitro and/or in vivo pentose transport activity; b) a polypeptide identical to an amino acid sequence shown in FIG. 1A, 2A, 3A, 4A, 5A, 6A, 7A or 8A, and; c) a fragment of a polypeptide as defined under a) or b) comprising a stretch of at least 100 continuous amino acids of an amino acid sequence shown in FIG. 1A, 2A, 3A, 4A, 5A, 6A, 7A or 8A and showing in vitro and/or in vivo pentose transport activity. Preferably, the polypeptide is selected from the group consisting of: a) a polypeptide having an amino acid sequence showing at least 80% identity with an amino acid sequence shown in FIG. 1A, 2A, 3A or 4A and showing in vitro and/or in vivo pentose transport activity; b) a polypeptide identical to an amino acid sequence shown in FIG. 1A, 2A, 3A or 4A, and; c) a fragment of a polypeptide as defined under a) or b) comprising a stretch of at least 100 continuous amino acids of an amino acid sequence shown in FIG. 1A, 2A, 3A or 4A and showing in vitro and/or in vivo pentose transport activity. More preferably, said polypeptide has L-arabinose and/or D-xylose transport activity. In one embodiment, pentose transport activity is L-arabinose transport activity. In another embodiment, pentose transport activity is D-xylose transport activity.

Xylose and arabinose transporters are known in the art. For example, Leandro et al. (2006 Biochem. J. 395:543-549) disclose two glucose/xylose transporter genes from the yeast Candida intermedia. Various reports are available on modifying the growth of fungi on pentoses, such as those by Bengtsson et al. (2009, Biotechnol. Biofuels 5;2:9) Krahulec et al. (2009 Biotechnol. J. 4:684-694), and Rundquist et al. (2009, Appl. Microbiol. Biotechnol. 82: 123-130). WO2008/080505 relates to arabinose transporters from the yeast Pichia stipitis and uses thereof in the production of biochemicals from biomass. WO2009/008756 discloses host cells transformed with a nucleic acid sequence encoding a specific L-arabinose transporter from yeast and the use of the host cell in the production of biofuels. WO2007/018442 relates to a Candida intermedia gene encoding an active transporter for xylose and modified yeast cells expressing the gene. However, the specific pentose transporters according to the present invention are not described or suggested in the art. In one embodiment, the polypeptide comprises a fragment of at least 200 or 300 continuous amino acids of a sequence shown in FIG. 1A, 2A, 3A, 4A, 5A, 6A, 7A or 8A. The fragment is characterized in that it displays in vitro and/or in vivo pentose transport activity, in particular arabinose or xylose transport activity. Pentose transport activity can be readily determined by methods known in the art. For example, it involves the use of radiolabelled (e.g. ¹⁴C) pentose and/or hexose substrates. See Walsh et al. (1994 J. Bacteriol. 176, 953-958).

Preferably, the polypeptide sequence shows at least 90%, preferably at least 95% identity with an amino acid sequence shown in FIG. 1A, 2A, 3A, 4A, 5A, 6A, 7A or 8A and showing in vitro and/or in vivo pentose transport activity. More preferably, the sequence is 96, 97, 98 or 99% identical to one of said sequences. In a specific aspect, the invention provides a polypeptide having a sequence identical to an amino acid sequence shown in FIG. 1A, 2A, 3A, 4A, 5A, 6A, 7A or 8A. The polypeptide may originate from any micro-organism, preferably a (filamentous) fungus, such as Aspergillus niger or another (industrially used) fungus e.g. selected from other Aspergillus species., Neurospora crassa, Magnaporthe grisea, Trichoderma species, Penicillium species, Fusarium species, Chrysosporium lucknowensis (Cl), Talaromyces sp., Thermomyces sp, Humicola sp. Saccharomyces species, Kluyveromyces sp., Hansenula sp., Pichia sp. and Yarrowia sp.

Also encompassed are variant or mutant polypeptides comprising one or more amino acid alterations (e.g. deletion, substitution and/or insertion) which do essentially keep the transport activity intact. In one embodiment, the variant comprises one or more conservative amino acid substitutions. Of course, activating mutations are of special interest.

A further aspect relates to a fusion protein comprising as a first fragment a transporter polypeptide described herein above and as a second fragment a heterologous polypeptide of interest. The first fragment can be located N- or C-terminally from the second fragment. Exemplary polypeptides of interest include sugar sensors, signaling pathway components, pentose converting metabolic enzymes (positioned intracellularly) and targeting sequences. In one embodiment, the transporter peptide is provided with a sequence selected from the group consisting of plasma membrane targeting sequences, and sequences increasing the turnover at the plasma membrane and sequences improving the proper localization in the hyphae. Also provided is an antibody or functional fragment thereof, capable of selectively binding to a pentose transporter of the invention. The skilled person will be able to generate such antibody (fragment) using methods known in the art. See for example “Antibodies: A Laboratory Manual” by Ed Harlow, Cold Spring Harbor Laboratory; David Lane, Imperial Cancer Research Fund Laboratories; ISBN 978-087969314-5.

A polypeptide of the invention can be provided using an isolated nucleic acid sequence disclosed herein. The nucleic acid sequence is typically a cDNA sequence. In one embodiment, there is provided an arabinose transporter gene that is at least 85% homologous to An08g01720 (FIG. 4B), An03g01620 (FIG. 3B) or An11g01100 (FIG. 2B). In another embodiment, there is provided a xylose transporter gene showing at least 85% identity to An06g00560 (FIG. 1B), An02g08230 (FIG. 7B), An07g00780 (FIG. 5B) or An13g02590 (6B). In one embodiment, the nucleic acid sequence is at least 85%, preferably at least 90% identical to a nucleic acid sequence shown in FIG. 1B, 2B, 3B, 4B, 5B, 6B, 7B or 8B. More preferably, the sequence is at least 91, 92, 93, 94, 95 96, 97, 98, or 99% identical. In a specific aspect, the nucleic acid sequence consists of a nucleic acid sequence shown in FIG. 1B, 2B, 3B, 4B, 5B, 6B, 7B or 8B.

The nucleic acid can be part of a larger nucleic acid molecule, for example an expression vector. Expression vectors allowing for expression of the encoded pentose transporter in a host cell, e.g. yeast host cell, are preferred. For example, pRS series plasmids (Silkorski R S, Hieter P 1989 A system of shuttle vectors and yeast host strains designed for efficient manipulation of DNA in Saccharomyces cerevisiae), pYES series plasmids (Invitrogen, Carlsbad Calif., USA), or pYEX series vectors (Clontech, CA, USA) may be used. The vector may contain one or more conventional elements, for example antibiotic resistance marker(s), transcriptional enhancers, and the like known to a skilled person in the art.

As will be understood, the pentose transporters provided herein and their encoding genes have a number of biotechnological and industrial applications. Homologous expression allows for modification of pentose uptake/utilization in A. niger by gene disruption or overexpression. The usage of efficient promoters such as the glucoamylase, endoxylanase, glyceraldehyde-triphosphate and other promoters known by skilled persons in the art can be used as promoters fit for expression under optimal process conditions. Heterologous expression of a transporter gene, in a manner similar to homologous expression, in a host cell other than A. niger can lead to enhanced pentose (L-arabinose, D-xylose) uptake and improved pentose utilization e.g. in biofuel production or any other type of application.

A vector encoding and allowing for expression of a pentose transporter disclosed herein is advantageously used to alter pentose uptake/utilization of a host cell. In one embodiment, the invention relates to a genetically engineered host cell provided with an isolated nucleic acid (preferably being part of a vector) encoding a polypeptide selected from the group consisting of a) a polypeptide having an amino acid sequence showing at least 80% identity with an amino acid sequence shown in FIG. 1A, 2A, 3A, 4A, 5A, 6A, 7A or 8A and showing in vitro and/or in vivo pentose transport activity; b) a polypeptide identical to an amino acid sequence shown in FIG. 1A, 2A, 3A, 4A, 5A, 6A or 7A, and; c) a fragment of a polypeptide as defined under a) or b) comprising a stretch of at least 100 continuous amino acids of an amino acid sequence shown in FIG. 1A, 2A, 3A, 4A, 5A, 6A or 7A and showing in vitro and/or in vivo pentose transport activity. In particular, it provides a recombinant host cell comprising an isolated nucleic acid sequence encoding a polypeptide as defined above, and wherein the host cell furthermore comprises at least one nucleic acid molecule encoding an enzyme involved in the metabolism of at least one pentose, like arabinose and/or xylose.

The host cell can be any suitable pro- or eukaryotic organism. In one embodiment, it is a fungal host cell. Preferably, the host cells are yeast cells and filamentous fungi, like Saccharomyces cerevisiae and Aspergillus niger. Other host cells of interest include Aspergillus species, Trichoderma species, Penicillium species, Fusarium species, also the ascomycetous fungus Chrysosporium lucknowense Cl, Saccharomyces species, Kluyveromyces sp., Hansenula sp., Pichia sp. and Yarrowia sp. Additional useful cells include basidiomycetes, for example a Trametes sp. such as T. versicolor.

In a specific embodiment, the invention also provides a fungal host cell, preferably a filamentous fungus, which is genetically modified to reduce the expression of at least one gene encoding a polypeptide according to the invention. This can be achieved by deletion or disruption of the corresponding gene, for instance by homologous recombination.

A host cell can be provided with further additional components, like at least one nucleic acid molecule encoding a protein involved in pentose metabolism, in particular the metabolism of xylose and/or arabinose. Examples include L-ribulokinase, L-ribulose-5-P 4-epimerase and L-arabinose-isomerase. Preferably, the nucleic acid molecule encodes a protein involved in the bacterial metabolism of arabinose and/or xylose. One or more of the E. coli araBAD operon encoding enzymes are suitably used. Other useful enzymes for heterologous expression include (P)H-dependent xylose reductase (XR) and NAD+-dependent xylitol dehydrogenase (XDH) from Pichia stipitis, bacterial xylose isomerase (XI) genes and xylulokinase.

The invention also relates to a method for converting a lignocellulosic crude carbon source into a value-added compound, comprising culturing a host cell as described herein above in the presence of said crude carbon source and allowing for expression of the pentose transporter. In particular, it provides a method for converting at least part of a lignocellulosic crude carbon source into a value-added compound, comprising culturing a host cell in the presence of said crude carbon source, the host cell expressing a nucleic acid sequence encoding a polypeptide selected from the group consisting of a) a polypeptide having an amino acid sequence showing at least 80% identity with an amino acid sequence shown in FIG. 1A, 2A, 3A, 4A, 5A, 6A, 7A or 8A, preferably FIG. 1A, 2A, 3A or 4A, and showing in vitro and/or in vivo pentose transport activity, b) a polypeptide identical to an amino acid sequence shown in FIG. 1A, 2A, 3A, 4A, 5A, 6A, 7A or 8A, preferably FIG. 1A, 2A, 3A or 4A, and c) a fragment of a polypeptide as defined under a) or b) comprising a stretch of at least 100 continuous amino acids of an amino acid sequence shown in FIG. 1A, 2A, 3A, 4A, 5A, 6A, 7A or 8A, preferably FIG. 1A, 2A, 3A or 4A, and showing in vitro and/or in vivo pentose transport activity.

Preferably, the ligocellulosic crude carbon source comprises pectin and/or hemicellulose. The crude carbon source preferably comprises arabinan, arabinogalactan, xylan and/or xyloglucan, preferably arabinose and/or xylose, more preferably L-arabinose and/or D-xylose, or any combination thereof, usually in the presence of hexoses (mainly glucose). Arabinan is a polysaccharide that is mostly a polymer of arabinose. Xylan (CAS number:9014-63-5) is a generic term used to describe a wide variety of highly complex polysaccharides that are found in plant cell walls and some algae. Xylans are polysaccharides made from units of xylose. Plant cell wall xylans differ strongly in the number and composition of side chains, depending on the plant species, but many contain arabinose attached to the xylose backbone. Xylan is found in the cell walls of some green algae, especially macrophytic siphonous genera, where it replaces cellulose. Similarly, it replaces the inner fibrillar cell-wall layer of cellulose in some red algae. Xylan is one of the foremost anti-nutritional factors in commonly used feedstuff raw materials. Xyloglucan is a hemicellulose which occurs in the primary cell wall of all vascular plants. In many dicotyledonous plants, it is the most abundant hemicellulose in the primary cell wall. Xyloglucan has a backbone of β1→4-linked glucose residues most of which are substituted with 1-6 linked xylose sidechains. The xylose residues are often capped with a galactose residue sometimes followed by a fucose residue, but can also be capped with arabinose. The specific structure of xyloglucan varies among plant families. Arabinogalactan is a mixed polysaccharide consisting of arabinose and galactose of which the ratio differs depending on the plant species.

Advantageously, the crude carbon source is typically a plant biomass or a composition derived there from, like hemicellulose hydrolysate. For example, the lignocellulosic crude carbon source may be selected from the group consisting of plant biomass, herbaceous material, agricultural residue, forestry residue, municipal solid waste, waste paper, and pulp and paper mill residue. In some embodiments, the lignocellulosic material is distiller's dried grains or distiller's dried grains with solubles. In some embodiments, the distiller's dried grains or distiller's dried grains with solubles are derived from corn.

The methods and host cells of the invention are advantageously applied in processes involving the co-utilization of pentoses with glucose. i.e. simultaneous utilization of glucose and xylose, the simultaneous utilization of glucose and arabinose or the simultaneous utilization of glucose, xylose and arabinose. This can be achieved by using a specific promoter to regulate the expression of one or more of the novel pentose transporters. It can be advantageous to provide the host cell with at least one gene encoding a hexose (e.g. glucose) transporter under the control of a specific promoter which may or may not be the same promoter as that controlling expression of the pentose transporter(s). Suitable promoters include constitutive or inducible promoters (expression under specific conditions). This embodiment is of particular interest in view of yeast strains which can use pentoses but use glucose/xylose/arabinose sequentially. The process time can be sped up if the carbon sources were used simultaneously, this would increase the efficiency of the process and decrease costs. The growth substrate can be any mixture of the pure sugars (hexose and pentose) or a substrate containing both glucose and the pentose(s), in particular those referred to herein above. Hence, the invention also provides a host cell comprising one or more of the novel pentose transporters, furthermore comprising at least one hexose transporter, preferably a glucose transporter. In one specific aspect, the pentose and hexose transporters are placed under the control of the same promoter. In another specific aspect, the pentose and hexose transporters are placed under the control of a distinct promoter.

The value-added compound can be any desirable or useful biochemical, like a biofuel, an organic acid, a proteinaceous substance, a sterol, and the like. Preferably, it is a biofuel, more preferably bioethanol. The ethanol yield and productivity can be improved by (heterologous) expression of a pentose transporter of the invention since it leads to an increased metabolic flux and consequent ethanol production. Therefore, the invention relates also to a method for providing bioethanol, comprising the expression of a nucleic acid according to the invention in a host cell which uses pentose and which expresses at least one of the novel pentose transporters shown in FIG. 1.

Also provided is the use of a polypeptide, a nucleic acid molecule, an expression vector and/or a host cell according to the invention to improve pentose uptake and/or utilization by a host cell, preferably a fungal host cell like yeast. In a preferred embodiment, the host cell is a recombinant industrial Saccharomyces cerevisiae strain e.g. an Ethanol red recombinant. In another preferred embodiment, it is a filamentous fungus. The pentose is arabinose, preferably L-arabinose, or xylose, preferably D-xylose.

In one embodiment, the invention provides the use of An06g00560 and/or An08g01720, or a host cell expressing the gene(s) or functional homologs thereof to enhance L-arabinose uptake and/or L-arabinose utilization. Preferably, An08g01720 is used in view of its low Km. 4.9 mM compared to 75 mM). In another specific embodiment, the invention provides the use of An03g01620, An03g02190 and/or An11g01100, or a host cell expressing the gene(s) or functional homologs thereof to enhance L-arabinose and/or D-xylose uptake and/or utilization. An11g01100 was found to preferentially transport xylose while An03g01620 would preferentially transport arabinose. Since An11g01100 has a higher affinity for D-xylose than L-arabinose, it is of particular use for increasing D-xylose uptake and/or utilization. In contrast, An03g01620 and An03g02190 have a higher affinity for L-arabinose). However, both would be suitable where either or both sugars are present

Furthermore, the invention provides a recombinant host cell comprising an isolated nucleic acid (preferably being part of a vector) encoding a polypeptide selected from the group consisting of a) a polypeptide having an amino acid sequence showing at least 80% identity with an amino acid sequence shown in FIG. 1A or 4A and showing in vitro and/or in vivo L-arabinose transport activity; b) a polypeptide identical to an amino acid sequence shown in FIG. 1A or 4A, and; c) a fragment of a polypeptide as defined under a) or b) comprising a stretch of at least 100 continuous amino acids of an amino acid sequence shown in FIG. 1A or 4A and showing in vitro and/or in vivo L-arabinose transport activity. Preferably, said host cell furthermore comprises at least one nucleic acid molecule encoding a protein involved in L-arabinose metabolism. Examples include L-ribulokinase, L-ribulose-5-P 4-epimerase and L-arabinose-isomerase. Still further, the invention provides a recombinant host cell comprising an isolated nucleic acid (preferably being part of a vector) encoding a polypeptide selected from the group consisting of a) a polypeptide having an amino acid sequence showing at least 80% identity with an amino acid sequence shown in FIG. 2A, 3A or 8A and showing in vitro and/or in vivo L-arabinose and D-xylose transport activity; b) a polypeptide identical to an amino acid sequence shown in FIG. 2A, 3A or 8A, and; c) a fragment of a polypeptide as defined under a) or b) comprising a stretch of at least 100 continuous amino acids of an amino acid sequence shown in FIG. 2A, 3A or 8A and showing in vitro and/or in vivo L-arabinose and D-xylose transport activity. Preferably, said host cell furthermore comprises at least one nucleic acid molecule encoding a protein involved in L-arabinose and/or D-xylose metabolism, for instance selected from the group consisting of L-ribulokinase, L-ribulose-5-P 4-epimerase, L-arabinose-isomerase, E. coli araBAD operon encoding enzymes, NAD(P)H-dependent xylose reductase (XR), NAD+-dependent xylitol dehydrogenase (XDH) from Pichia stipitis, bacterial xylose isomerase (XI) genes and xylulokinase. In a specific aspect, the host cell comprises at least one (exogenous) nucleic acid molecule encoding a protein involved in L-arabinose and at least one (exogenous) nucleic acid molecule encoding a protein involved in D-xylose metabolism.

A further specific aspect relates to a recombinant host cell comprising an isolated nucleic acid (preferably being part of a vector) encoding a polypeptide selected from the group consisting of a) a polypeptide having an amino acid sequence showing at least 80% identity with an amino acid sequence shown in FIG. 2A and showing in vitro and/or in vivo D-xylose transport activity; b) a polypeptide identical to an amino acid sequence shown in FIG. 2A, and; c) a fragment of a polypeptide as defined under a) or b) comprising a stretch of at least 100 continuous amino acids of an amino acid sequence shown in FIG. 2A and showing in vitro and/or in vivo D-xylose transport activity. Preferably, said host cell furthermore comprises at least one nucleic acid molecule encoding a protein involved in D-xylose metabolism, for instance selected from the group consisting of L-ribulokinase, L-ribulose-5-P 4-epimerase, E. coli araBAD operon encoding enzymes, NAD(P)H-dependent xylose reductase (XR), NAD+-dependent xylitol dehydrogenase (XDH) from Pichia stipitis, bacterial xylose isomerase (XI) genes and xylulokinase.

### LEGEND TO THE FIGURES

FIG. 1: A) Amino acid sequence of the A. niger pentose transporter protein An06g00560; B) Nucleotide sequence of the An06g00560 gene.

FIG. 2: A) Amino acid sequence of the A. niger pentose transporter protein An11g01100; B) Nucleotide sequence of the An11g01100 gene.

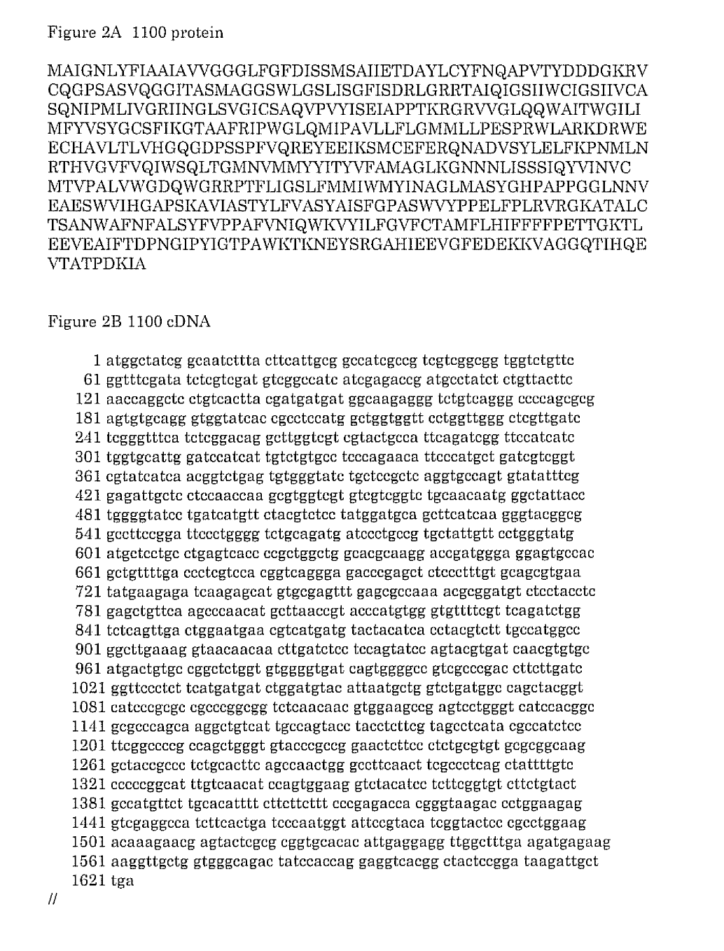

FIG. 3: A) Amino acid sequence of the A. niger pentose transporter protein An03g01620; B) Nucleotide sequence of the An03g01620 gene.

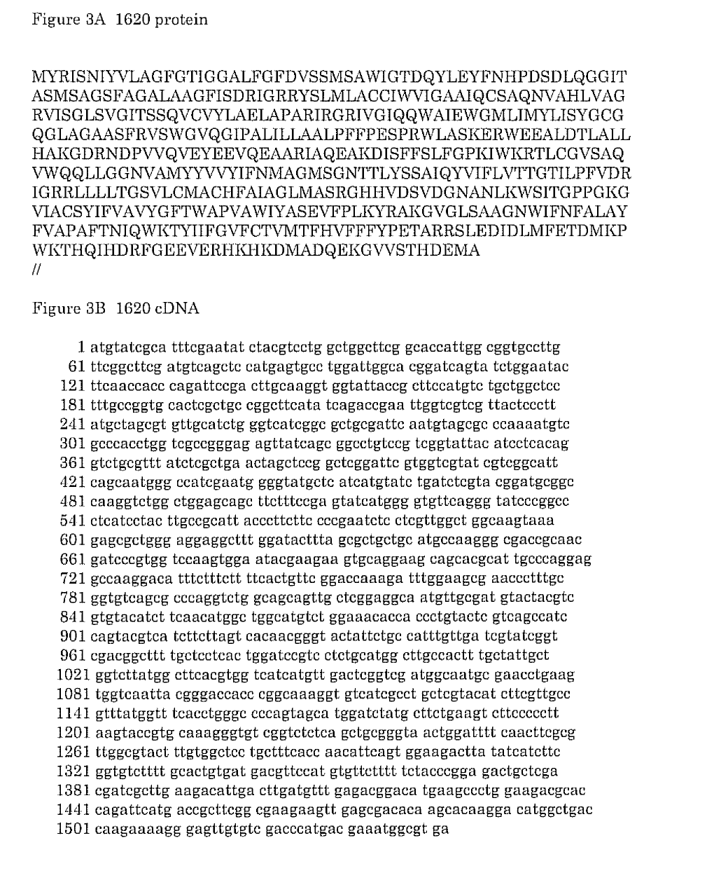

FIG. 4: A) Amino acid sequence of the A. niger pentose transporter protein An08g01720; B) Nucleotide sequence of the An08g01 720 gene.

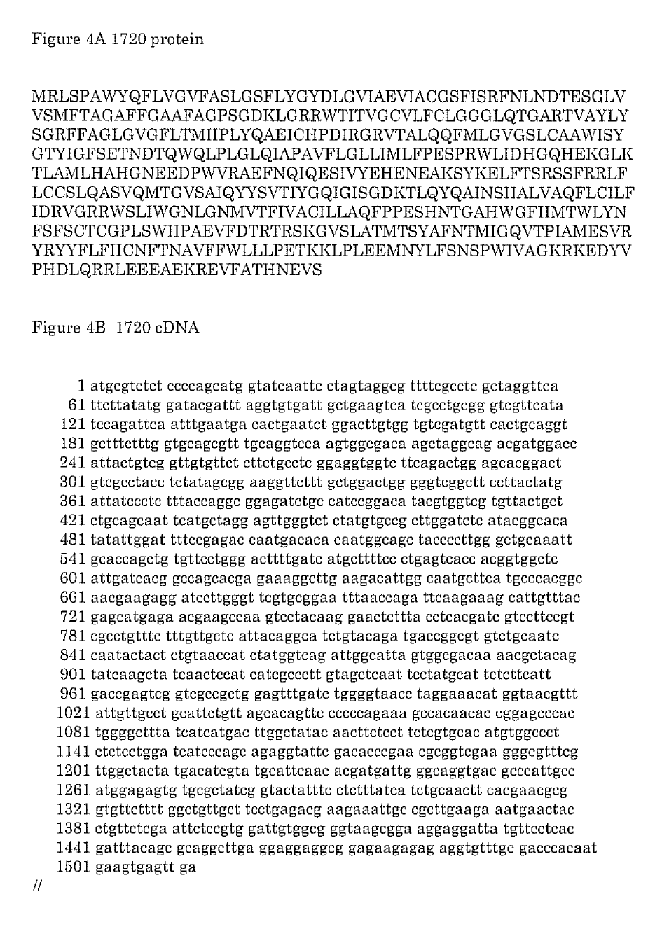

FIG. 5: A) Amino acid sequence of the A. niger pentose transporter protein An07g00780; B) Nucleotide sequence of the An07g00780 gene.

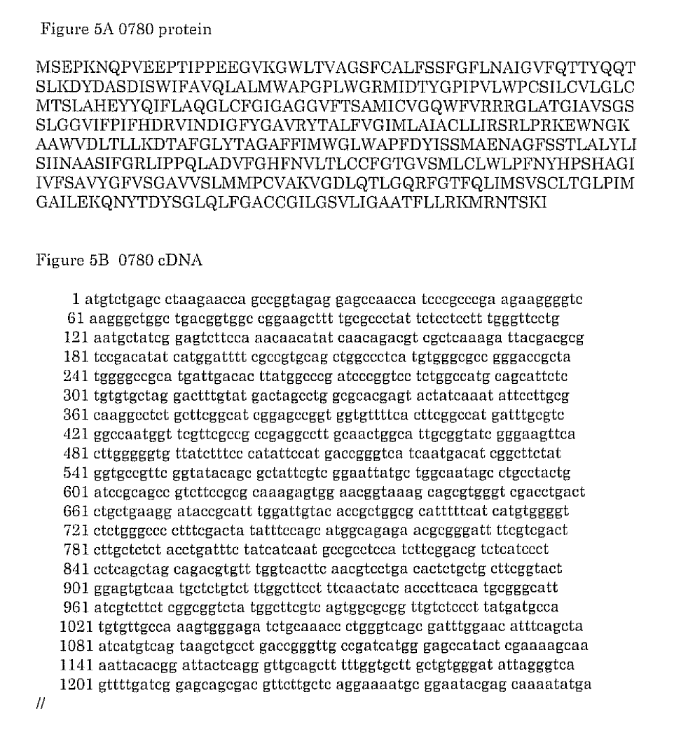

FIG. 6: A) Amino acid sequence of the A. niger pentose transporter protein An13g02590; B) Nucleotide sequence of the An13g02590 gene.

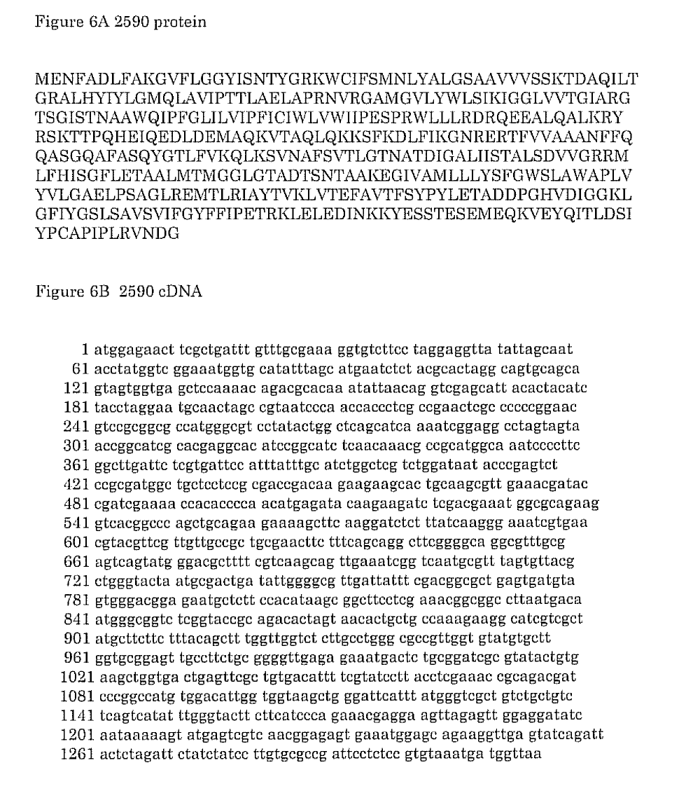

FIG. 7: A) Amino acid sequence of the A. niger pentose transporter protein An02g08230; B) Nucleotide sequence of the An02g08230 gene.

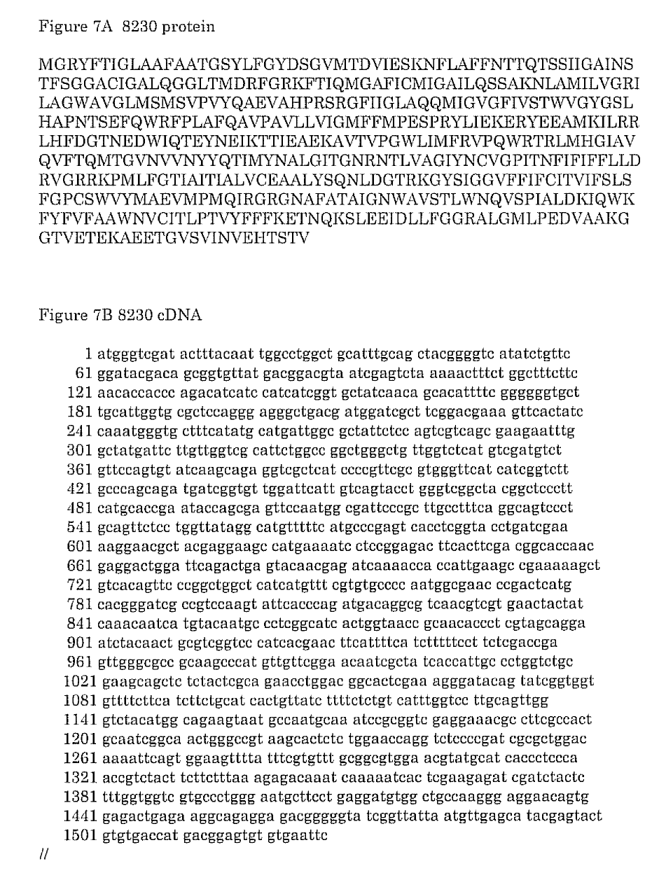

FIG. 8: A) Amino acid sequence of the A. niger pentose transporter protein An03g02190; B) Nucleotide sequence of the An03g02190 gene.

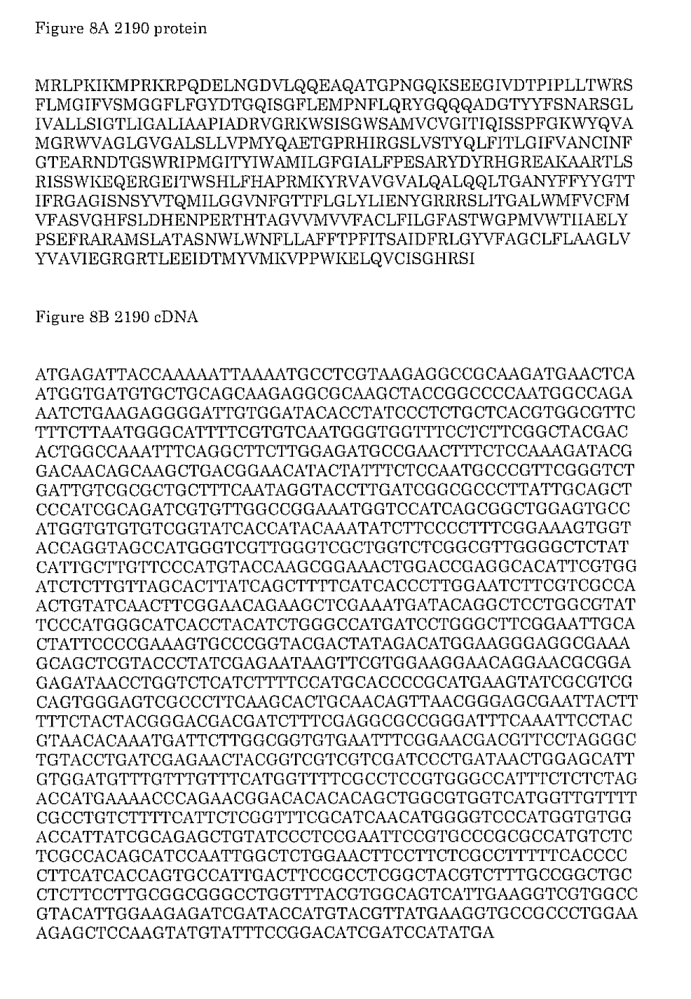

### EXPERIMENTAL SECTION

### Materials and Methods

### Strains and Growth Conditions

All A. niger strains used were derived from A. niger N400 (=CBS120.49) and are described in Table 1. Precultures were grown in complete medium [de Vries, R. P., K. Burgers, P. J. I. van de Vondervoort, J. C. Frisvad, R. A. Samson, and J. Visser. 2004. A new black Aspergillus species, A. vadensis, is a promising host for homologous and heterologous protein production. Appl. Environ. Microbiol, 70: 3954-3959.], pH 6.0, with 2% fructose, in a rotary shaker at 250 rev./min and 30° C. For the growth of strains with auxotrophic markers, the necessary supplements were added to the medium. After 16 h mycelium was harvested and washed with MM without carbon source, and aliquots of 1 gr (wet weight) mycelium were added to 50 ml MM with 25 mM L-arabinose or D-xylose and incubated for an additional 2 hours, before harvesting. The mycelium was harvested by suction over a filter, washed with MM without a carbon source, dried between paper and frozen in liquid nitrogen. The mycelium samples were stored at −70° C.

Yeast strain EB.VW4000 was grown on YP [1% (w/v) Bacto yeast extract/2% (w/v) Bacto peptone]with 2% (w/v) maltose at 30° C. (ref strain). Other S. cerevisiae strains used were derived from strain EB.VW4000, and were transformed with plasmids based on pYEX-BX (Clontech). Plasmid transformations of yeast cells were carried out according to the quick and easy TRAFO protocol [TRAFO reference]. Yeast strains were grown at 30° C. in a rotary shaker at 250 rev./min, in YNB[0.67% (w/v) Difco yeast nitrogen base] supplemented with 0.1% (w/v) casamino acids and 0.2 mg/l tryptophan, 20 mg/l leucine, 20 mg/l histidine, 122 mg/l uridine. The carbon sources used were as stated in the text. Unless stated otherwise, 0.5 mM CuSO₄ was used to induce expression from the CUP1 promoter.

¹⁴C-D-xylose plate screens were carried out with the addition of x ¹⁴C-1-D-xylose to solid media (2% Difco agar). 10³ yeast cells were inoculated onto defined positions on a polycarbonate membrane and incubated at 30° C. for two days. In case any ¹⁴C-carbon dioxide was produced the plates were placed in a large sealed glass vessel which also contained NaOH based carbon trap. Autoradiograph using x film enabled the detection of colonies which had transported radiolabelled D-xylose.

### Molecular Biology Methods

Unless stated otherwise, general methods such as PCR, ligation, digestion, transformation of Escherichia coli (DHF5αF), plasmid DNA isolation and gel electrophoresis were performed according to standard procedures (Sambrook et al., 1989). Total RNA was isolated from powdered mycelium using TRIzol® reagent (Life Technologies), according to the supplier's instructions. For Northern-blot analysis 3 μg of total RNA was incubated with 3.3 μl of 6M glyoxal, 10 μl of DMSO and 2 μl of 0.1M sodium phosphate buffer, pH 7, in a total volume of 20 μl for 1 h at 50° C. to denature the RNA. The RNA samples were separated on a 1.5% agarose gel using 0.01M sodium phosphate buffer (pH 7) and transferred to Hybond-N filters (Amersham Biosciences) by capillary blotting. Washing of Southern blots was performed under stringent conditions with 30 mM NaCl, 3 mM sodium citrate and 0.5% (w/v) SDS at 68° C. DIG-labelling of probes, their hybridization and detection was performed according to the manufacturers instructions (Roche).

Yeast expression constructs were generated by PCR using cDNA libraries as the template. Oligonucleotides used are described in Table 2. cDNA libraries used were from a germination time course, and mycelia grown on L-arabinose or D-xylose prepared as described by VanKuyk et al. Biochem. J. (2004) 379, 375-383 and De Groot et al. (2007) Food Technol. Biotechnol. 45: 134-138. The germination time-course library was constructed using the CloneMiner cDNA library construction method (InVitrogen) according to the supplier's instructions. The conidiation library was constructed form mycelium that was pregrown for 16 hours in CM-glucose medium and transferred to an agar plate with a polycarbonate filter. Mycelium was either covered with a second polycarbonate filter to inhibit conidiation, or incubated without a second filter and grown for 8 or 27 h. For each library, equal amount of RNA from the different condition were pooled and used for the construction. cDNA were cloned into Donor Vector pDONOR222 to create the entry library. As gene An13g02590 could not be obtained from the cDNA libraries, it was amplified using Superscript® One-Step RT-PCr System (Invitrogen, Paisley, UK) from RNA. Products from duplicate PCRs were cloned into either pGEMT-easy (Promega, Wis., USA) or pJET (Fermentas, Ontario, Canada) in accordance to the manufacturer's instructions. Multiple clones from each reaction were sequenced (Macrogen, South Korea), and the sequences compared to the published A. niger genome sequences of strain NRRL3122 (CBS 513.88) and ATCC 1015 (Pel et al. 2007. Nat. Biotechnol. 25, 221-231, and Baker S E (2006, Med. Mycol. 44: Suppl 1:S17-21). Sequence comparison was used to identify PCR errors, strain differences, and unspliced introns. Genes which contained fully processed cDNAs with no PCR errors were cloned into pYEX-BX (Clontech, California. USA) for analysis in S. cerevisiae.

### Microarray Analysis

Biotin-labeled antisense cRNA was generated by labelling 20 or 2 g of total RNA with a BioArray high-yield RNA transcription labeling kit (ENZO) or an Affymetrix eukaryotic one-cycle target labeling and control reagent package, respectively. The quality of the cRNA was checked using the Agilent 2100 bioanalyzer. The labeled cRNA was hybridized to Affymetrix A. niger GeneChips (Affymetrix, Santa Clara, Calif.). The coding sequence of the annotated genome of CBS513.88 (13) was taken as the sequence template.

Oligonucleotide probes were designed with 600-bp fragments, starting from the 3′ end of the gene. The probe sets consist of 12 pairs (match and mismatch) of 25-bp oligonucleotide probes, which are scattered across the chip. Absolute values of expression were calculated from the scanned array by using Affymetrix GeneChip Operating System software after an automated process of washing and staining. Microarray Suite Affymetrix version 5.1 (Affymetrix Inc., Santa Clara, Calif.), Spotfire DecisionSite (Spotfire, Inc. Somerville, Mass.), Gene-Data Expressionist Analyst V Pro 2.0.18 (GeneData, Basel, Switzerland), and the R statistical environment (www.r-project.org) were used for data analyses. Arrays were hybridized with three independently obtained RNA samples of the peripheries of 7-day-old sandwiched cultures grown on maltose. Since the correlation between the samples was 0.982 and the average signal log ratio was found to be _{—}0.044, it was decided that all other hybridizations would be done with biological duplicates.

Affymetrix DAT files were processed using the Affymetrix GeneChip Operating System. The CHP files were generated from CEL files by using Affymetrix Global scaling normalization to a target intensity value of 100 (TGT-100).

### Functional Analysis

Complementation of the hexose transport defect of strain EB.VW4000 was assayed by measuring growth (OD590) using a Perkin Elmer X. Measurements were taken at 24 hour intervals. Excel (Microsoft, USA) was used to transform the individual data from 6-8 replicates of each condition into average OD590 and standard deviations which were graphed to enable strain comparisons.

For the sugar-transport experiments (zero trans-influx assays) yeast strains were grown for 16-20 h (approx. D600, 2.0). Cells were pelleted by centrifugation (10 min at 4000×g), washed in ice-cold 0.1M phosphate buffer (pH 6.5), and resuspended to give a 10% wet weight/volume suspension in 0.1M phosphate buffer (pH 6.5). Cells were kept on ice until required. Zero trans-influx of ¹⁴C-labelled D-glucose, D-fructose, D-mannose and D-xylose during a 5 s incubation at 30° C. was determined according to Walsh et al. For experiments done at pH 5, 0.1M phthalic acid (pH 5.0) was used instead of phosphate buffer. Enzfitter software (version 1.05; Biosoft) was used to determine the apparent kinetic parameters of the transport protein for the different monosaccharides by non-linear regression analysis.

Proton uptake during sugar transport was monitored by recording pH changes in yeast suspensions as described previously Serrano, R. (1977) Eur. J. Biochem. 80, 97-102; Santos, E (1982) Arch. Biochem. Biophys. 216, 625-660, using a Titralab model (Radiometer, Copenhagen, Denmark). The suspension was mixed using a magnetic stirrer at 30° C. pH was lowered to 5.0-5.1 by pulses of 10 mM HCl from commencement of the individual experiments.

### Transfer Experiment:

pre-culture: 16 h CM 2% fructose
transfer: appr. 1 gr (wet weight) mycelium to 50 ml MM with 25 mM L-arabinose or xylose

TABLE 1
Fungal strains used in this study

| Species | name | Description | genotype | reference |
|---|---|---|---|---|
| A. niger | N402 | wild type | | Bos et al. 1988 |
| A. niger | UU-A033.21 | araR | nicA1, leuA1, delta | Battaglia et al, |
| | | disruptant | argB::delta araR, pyrA6 | |
| A. niger | UU-A062.10 | xlnR | nicA1, leuA1, delta argB, | Battaglia et al, |
| | | disruptant | pyrA6::delta xlnR | |
| S. cerevisiae | EB.VW.4000 | Hexose | MATdeltaleu2-3, 112 | (Wieczorke et |
| | | transport | ura3-52 trp1-289 his3- | al. FEBS Lett. |
| | | minus strain | delta1 MAL2-8^{c} SUC2 | 1999 Dec |
| | | | hxt17delta | 31; 464(3): 123-8.) |
| | | | hxt13delta::loxP | |
| | | | hxt15delta::loxP | |
| | | | hxt16delta:delta:loxP | |
| | | | hxt14delta::loxP | |
| | | | hxt12delta::loxP | |
| | | | hxt9delta::loxP | |
| | | | hxt11delta::loxP | |
| | | | hxt10delta::loxP | |
| | | | hxt8delta::loxP | |
| | | | hxt514delta::loxP | |
| | | | hxt2delta::loxP | |
| | | | hxt367delta::loxP | |
| | | | gal2delta | |

TABLE 2
Oligonucleotides used in this study. Restriction sites used for
cloning are indicated in italics.

| gene | Oligo name | Sequence 5′-3′ |
|---|---|---|
| An08g01720 | An08g01720up_SalI | GTCGACATGCGTCTCTCCCCAGCATG |
| | An08g01720dw_SalI | ATATGTCGACTCAACTCACTTCATTGT |
| | | GGGTCG |
| An03g01620 | An03g01620up_BglII | ATATAGATCTATGTATCGCATTTCGAA |
| | | TATCTACG |
| | An03g01620dw_EcoRI | ATATGAATTCTCACGCCATTTCGTCAT |
| | | GG |
| An11g01100 | An11g01100up2_SmaI | CCCGGGCATCATGGCTATCGGCAA |
| | An11g01100dw_EcoRI | ATATGAATTCAAGCAATCTTATCCGGA |
| | | GTAG |
| An06g00560 | An06g00560up_BamHI | ATATGGATCCTCAACATGGGTATGGG |
| | | TGC |
| | An06g00560dw_NsiI | ATATATGCATTACGCCGAGGGAGGAG |
| | | TC |
| An13g02590 | An13g02590up1_BamHI | ATATGGATCCAGAATGCTCATTTTCAC |
| | | TACCG |
| | An13g02590up2_BamHI | ATATGGATCCAAGCTATGGAGAACTTC |
| | | GCTG |
| | An13g02590dw2_EcoRI | GAATTCATATCAGTTTTGTACATCCGC |
| | | C |
| | An13g02590dw_EcoRI | ATATGAATTCTTTAACCATCATTTACA |
| | | CGGAG |
| An02g08230 | An02g08230up_BamHI | GGATCCTATCCATCGGTGTCTCAAGAT |
| | An02g08230dw_EcoRI | GAATTCACACACTCCGTCATGGTCAC |
| An07g00780 | An07g00780up1_BamHI | ggatccAACCATGTCTGAGCCTAAGA |
| | An07g00780dw1_EcoRI | gaattcGCGGGATAGCCACCA |

### Effect of Pentose Transporters on Yeast Growth

Yeast strains provided with an exemplary novel pentose transporter were studied with respect to the effect of growth in the presence of pentose sugars. As shown in Table 3, it was observed that S. cerevisiae strains expressing the 0560 or the 1100 transporter showed a reduced growth in the presence of both L-arabinose and D-xylose. We believe the reduced growth is due to a toxic effect of (unregulated) intracellular accumulation of pentose sugars (or their metabolites) by a pentose non-utilizing S. cerevisiae strain. Interestingly, an effect of both sugars was observed to some degree for both proteins.

TABLE 3
Results showing the toxic effect of enhanced pentose uptake in host cells
provided with a gene encoding a novel pentose transporter.

| Sugar | | | STRAIN | | |
|---|---|---|---|---|---|
| maltose | L-arabinose | D-xylose | Control | 0560 | 1100 |
| 50 mM | | | ++++ | ++++ | ++++ |
| 50 mM | 25 mM | | ++++ | + | +++ |
| 50 mM | 50 mM | | ++++ | +/− | ++ |
| 50 mM | 100 mM | | ++++ | +/− | + |
| 50 mM | 500 mM | | ++++ | − | +/− |
| 50 mM | | 25 mM | ++++ | +++ | ++ |
| 50 mM | | 50 mM | ++++ | ++ | + |
| 50 mM | | 100 mM | ++++ | ++ | + |
| 50 mM | | 500 mM | ++++ | ++ | + |

Growth medium used was YNB + 2% agar + 0.5 mM CuSO₄.
Concentrations of sugars are as indicated.
++++ = very good growth,
+++ = good growth,
++ = poor growth,
+ = very poor growth,
+/− = growth barely visible,
− = no growth.

### Pentose Transport Activity Measurements

For the sugar-transport experiments (zero trans-influx assays) yeast strains were grown for 16-20 h (approx. D600, 2.0). Cells were pelleted by centrifugation (10 min at 4000 g), washed in ice-cold 0.1M phosphate buffer (pH 6.5), and resuspended to give a 10% wet weight/volume suspension in 0.1M phosphate buffer (pH 6.5). Cells were kept on ice until required. Zero trans-influx of 14C-labelled L-arabinose or D-xylose during a 5 s incubation at 30° C. was determined according to Walsh et al. (1994, J. Bacteriol. 176, 953-958). Results are shown in Table 4 below.

TABLE 4
Km values for transporter of arabinose and xylose by novel pentose
transporters based on data obtained in the zero-trans-influx assays.

| GENE | ARABINOSE | XYLOSE |
|---|---|---|
| An06g00560 | 75 mM | TND |
| An03g01620 | 7.5 mM | 23 mM |
| An03g02190 | 2.6 mM | 9 mM |
| An11g01100 | 200 mM | 138 mM |
| An08g01720 | 4.9 mM | TND |

TND = transport not detected

As is clear from the Km values of Table 4, An06g00560 and An08g01720 both transport L-arabinose but not D-xylose at detectable levels, thus these two transporters are specific for L-arabinose. An08g01720 has an approximately 15-fold higher affinity for L-arabinose than An06g00560 (4.9 mM compared to 75 mM). An03g01620, An03g02190 and An11g01100 all transport both pentose sugars tested. An11g01100 has a higher affinity for D-xylose than L-arabinose, although the Km values obtained for both sugars are in the same order of magnitude (i.e. 100-200 mM). An03g01620 and An03g02190 have a higher affinity for L-arabinose (Km in the 1-10 mM range) than D-xylose for which the respective Km values are approximately 3 times higher.

Thus with Km values for L-arabinose ranging from 2.6 mM to 200 mM and for D-xylose ranging from 9 mM to 138 mM, this set of transporters is able to transport the pentose sugars L-arabinose and D-xylose over a wide range of concentrations.

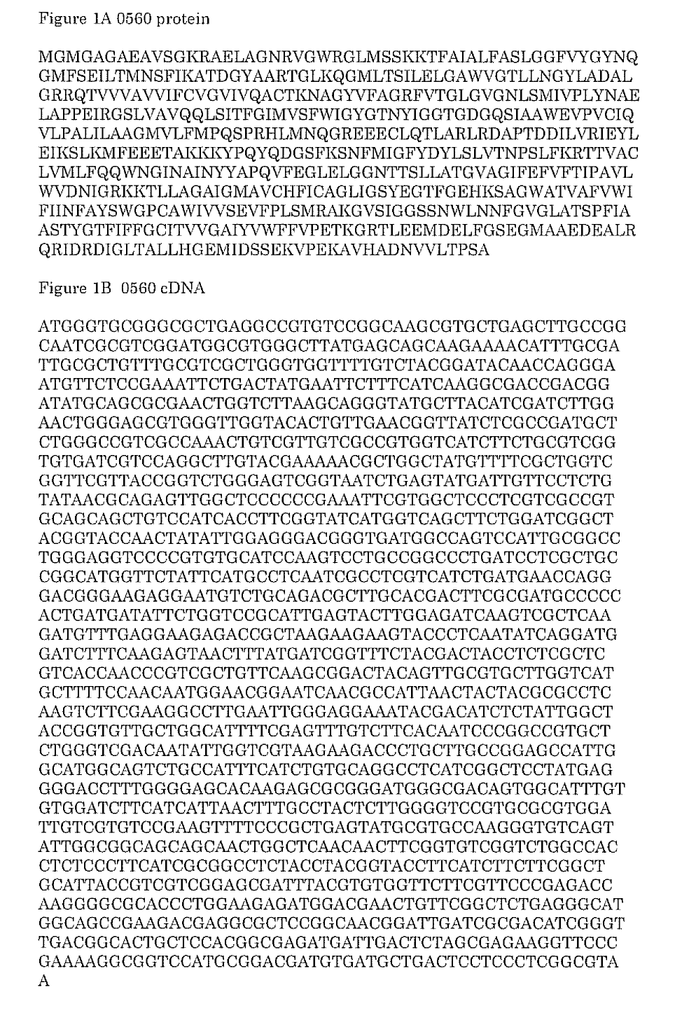

1. A method for converting at least part of a lignocellulosic crude carbon source into a value-added compound, comprising culturing a host cell in the presence of said crude carbon source, the host cell expressing a nucleic acid sequence encoding a polypeptide selected from the group consisting of
a) a polypeptide having an amino acid sequence showing at least 80% identity with an amino acid sequence shown in FIG. 1A, 2A, 3A, 4A, 5A, 6A, 7A or 8A and showing in vitro and/or in vivo pentose transport activity,
b) a polypeptide identical to an amino acid sequence shown in FIG. 1A, 2A, 3A, 4A, 5A, 6A, 7A or 8A and
c) a fragment of a polypeptide as defined under a) or b) comprising a stretch of at least 100 continuous amino acids of an amino acid sequence shown in FIG. 1A, 2A, 3A, 4A, 5A, 6A, 7A or 8A and showing in vitro and/or in vivo pentose transport activity.

2. The method according to claim 1, wherein the host cell expresses a fragment of at least 200, preferably 300, continuous amino acids of a sequence shown in FIG. 1A, 2A, 3A, 4A, 5A, 6A, 7A or 8A.

3. The method according to claim 1, wherein the crude carbon source comprises pectin and/or hemicellulose.

4. The method according to any claim 3, wherein the crude carbon source comprises arabinan, arabinogalactan, xylan or xyloglucan.

5. The method according to claim 1, wherein the crude carbon source is selected from the group consisting of plant biomass, herbaceous material, agricultural residue, forestry residue, municipal solid waste, waste paper, and pulp and paper mill residue.

6. The method according to claim 1, wherein the value-added compound is a biofuel.

7. The method according to claim 1, wherein the host cell comprises at least one nucleic acid molecule encoding an enzyme involved in the metabolism of arabinose or xylose.

8. The method according to claim 7, wherein the host cell comprises at least one gene encoding an enzyme selected from the group consisting of L-ribulokinase, L-ribulose-5-P 4-epimerase, L-arabinose-isomerase, E. coli araBAD operon encoding enzymes, NAD(P)H-dependent xylose reductase (XR), NAD+-dependent xylitol dehydrogenase (XDH), xylose isomerase (XI) and xylulokinase.

9. A recombinant host cell comprising an isolated nucleic acid sequence encoding a polypeptide as defined in claim 1, and wherein the host cell comprises at least one nucleic acid molecule encoding an enzyme involved in the metabolism of at least one pentose.

10. The host cell according to claim 9, wherein the nucleic acid sequence is at least 90% identical to a nucleic acid sequence shown in FIG. 1B, 2B, 3B, 4B, 5B, 6B, 7B or 8B.

11. The host cell according to claim 9, wherein the nucleic acid sequence is at least 95% identical to a nucleic acid sequence shown in FIG. 1B, 2B, 3B or 4B.

12. The host cell according to claim 9, comprising at least one gene encoding an enzyme selected from the group consisting of L-ribulokinase, L-ribulose-5-P 4-epimerase, L-arabinose-isomerase, E. coli araBAD operon encoding enzymes, NAD(P)H-dependent xylose reductase (XR), NAD+-dependent xylitol dehydrogenase (XDH), xylose isomerase (XI) and xylulokinase.

13. The host cell according to claim 9, wherein the host cell is a fungus.

14. The host cell according to claim 13, being a Saccharomyces cerevisiae or Aspergillus niger host cell.

15. A method of improving the uptake or utilization of at least one pentose by a host cell comprising the host cell expressing a polypeptide selected from the group consisting of
a) a polypeptide having an amino acid sequence showing at least 80% identity with an amino acid sequence shown in FIG. 1A, 2A, 3A, 4A, 5A, 6A, 7A or 8A, preferably FIG. 1A, 2A, 3A or 4A, and showing in vitro and/or in vivo pentose transport activity,
b) a polypeptide identical to an amino acid sequence shown in FIG. 1A, 2A, 3A, 4A, 5A, 6A, 7A or 8A, preferably FIG. 1A, 2A, 3A or 4A, and
c) a fragment of a polypeptide as defined under a) or b) comprising a stretch of at least 100 continuous amino acids of an amino acid sequence shown in FIG. 1A, 2A, 3A, 4A, 5A, 6A, 7A or 8A, preferably FIG. 1A, 2A, 3A or 4A, and showing in vitro and/or in vivo pentose transport activity,
in the presence of the least one pentose.

16. The method according to claim 15, wherein the pentose is arabinose.

17. The method according to claim 15, wherein the host cell is a yeast cell or filamentous fungus.

18. The method according to claim 16, wherein the host cell is selected from the group consisting of Aspergillus species, Trichoderma species, Saccharomyces species, Chrysosporium lucknowense Cl, Kluyveromyces sp., Hansenula sp., Pichia sp. and Yarrowia sp.

19. The method according to claim 18, wherein the host cell is a Saccharomyces cerevisiae or Aspergillus niger host cell.

20. The method according to any claim 3, wherein the crude carbon source comprises arabinose or xylose.

21. The method according to any claim 3, wherein the crude carbon source comprises L-arabinose or D-xylose.

22. The method according to claim 1, wherein the value-added compound is a bioethanol.

23. The host cell of claim 9 wherein the at least one pentose is arabinose or xylose.

24. The host cell according to claim 9, wherein the nucleic acid sequence is at least 95% identical to a nucleic acid sequence shown in FIG. 1B, 2B, 3B, 4B, 5B, 6B, 7B or 8B.

25. The host cell according to claim 9, wherein the host cell is a fungus selected from the group consisting of Aspergillus species, Trichoderma species, Saccharomyces species, Chrysosporium lucknowense Cl, Kluyveromyces sp., Hansenula sp., Pichia sp. and Yarrowia sp.

26. The method according to claim 15, wherein the pentose is L-arabinose or D-xylose.


SEQUENCE LISTING (32 sequences)

SEQ ID NO: 1 - DNA, 26 bp, Artificial
  An08g01720up_SalI oligonucleotide
gtcgacatgcgtctctccccagcatg

SEQ ID NO: 2 - DNA, 33 bp, Artificial
  An08g01720dw_SalI oligonucleotide
atatgtcgactcaactcacttcattgtgggtcg

SEQ ID NO: 3 - DNA, 35 bp, Artificial
  An03g01620up_BglII oligonucleotide
atatagatctatgtatcgcatttcgaatatctacg

SEQ ID NO: 4 - DNA, 29 bp, Artificial
  An03g01620dw_EcoRI oligonucleotide
atatgaattctcacgccatttcgtcatgg

SEQ ID NO: 5 - DNA, 24 bp, Artificial
  An11g01100up2_SmaI oligonucleotide
cccgggcatcatggctatcggcaa

SEQ ID NO: 6 - DNA, 31 bp, Artificial
  An11g01100dw_EcoRI oligonucleotide
atatgaattcaagcaatcttatccggagtag

SEQ ID NO: 7 - DNA, 29 bp, Artificial
  An06g00560up_BamHI oligonucleotide
atatggatcctcaacatgggtatgggtgc

SEQ ID NO: 8 - DNA, 28 bp, Artificial
  An06g00560dw_NsiI oligonucleotide
atatatgcattacgccgagggaggagtc

SEQ ID NO: 9 - DNA, 32 bp, Artificial
  An13g02590up1_BamHI oligonucleotide
atatggatccagaatgctcattttcactaccg

SEQ ID NO: 10 - DNA, 31 bp, Artificial
  An13g02590up2_BamHI oligonucleotide
atatggatccaagctatggagaacttcgctg

SEQ ID NO: 11 - DNA, 28 bp, Artificial
  An13g02590dw2_EcoRI oligonucleotide
gaattcatatcagttttgtacatccgcc

SEQ ID NO: 12 - DNA, 32 bp, Artificial
  An13g02590dw_EcoRI oligonucleotide
atatgaattctttaaccatcatttacacggag

SEQ ID NO: 13 - DNA, 27 bp, Artificial
  An02g08230up_BamHI oligonucleotide
ggatcctatccatcggtgtctcaagat

SEQ ID NO: 14 - DNA, 26 bp, Artificial
  An02g08230dw_ EcoRI oligonucleotide
gaattcacacactccgtcatggtcac

SEQ ID NO: 15 - DNA, 26 bp, Artificial
  An07g00780up1_BamHI oligonucleotide
ggatccaaccatgtctgagcctaaga

SEQ ID NO: 16 - DNA, 21 bp, Artificial
  An07g00780dw1_EcoRI oligonucleotide
gaattcgcgggatagccacca

SEQ ID NO: 17 - DNA, 1701 bp, Aspergillus niger
  CDS (1)..(1701)
atgggtgcgggcgctgaggccgtgtccggcaagcgtgctgagcttgccggcaatcgcgtcggatggcgtgggcttatgagcagcaagaaaacatttgcgattgcgctgtttgcgtcgctgggtggttttgtctacggatacaaccagggaatgttctccgaaattctgactatgaattctttcatcaaggcgaccgacggatatgcagcgcgaactggtcttaagcagggtatgcttacatcgatcttggaactgggagcgtgggttggtacactgttgaacggttatctcgccgatgctctgggccgtcgccaaactgtcgttgtcgccgtggtcatcttctgcgtcggtgtgatcgtccaggcttgtacgaaaaacgctggctatgttttcgctggtcggttcgttaccggtctgggagtcggtaatctgagtatgattgttcctctgtataacgcagagttggctccccccgaaattcgtggctccctcgtcgccgtgcagcagctgtccatcaccttcggtatcatggtcagcttctggatcggctacggtaccaactatattggagggacgggtgatggccagtccattgcggcctgggaggtccccgtgtgcatccaagtcctgccggccctgatcctcgctgccggcatggttctattcatgcctcaatcgcctcgtcatctgatgaaccagggacgggaagaggaatgtctgcagacgcttgcacgacttcgcgatgcccccactgatgatattctggtccgcattgagtacttggagatcaagtcgctcaagatgtttgaggaagagaccgctaagaagaagtaccctcaatatcaggatggatctttcaagagtaactttatgatcggtttctacgactacctctcgctcgtcaccaacccgtcgctgttcaagcggactacagttgcgtgcttggtcatgcttttccaacaatggaacggaatcaacgccattaactactacgcgcctcaagtcttcgaaggccttgaattgggaggaaatacgacatctctattggctaccggtgttgctggcattttcgagtttgtcttcacaatcccggccgtgctctgggtcgacaatattggtcgtaagaagaccctgcttgccggagccattggcatggcagtctgccatttcatctgtgcaggcctcatcggctcctatgaggggacctttggggagcacaagagcgcgggatgggcgacagtggcatttgtgtggatcttcatcattaactttgcctactcttggggtccgtgcgcgtggattgtcgtgtccgaagttttcccgctgagtatgcgtgccaagggtgtcagtattggcggcagcagcaactggctcaacaacttcggtgtcggtctggccacctctcccttcatcgcggcctctacctacggtaccttcatcttcttcggctgcattaccgtcgtcggagcgatttacgtgtggttcttcgttcccgagaccaaggggcgcaccctggaagagatggacgaactgttcggctctgagggcatggcagccgaagacgaggcgctccggcaacggattgatcgcgacatcgggttgacggcactgctccacggcgagatgattgactctagcgagaaggttcccgaaaaggcggtccatgcggacgatgtgatgctgactcctccctcggcgtaa

SEQ ID NO: 18 - protein, 566 aa, Aspergillus niger
MGAGAEAVSGKRAELAGNRVGWRGLMSSKKTFAIALFASLGGFVYGYNQGMFSEILTMNSFIKATDGYAARTGLKQGMLTSILELGAWVGTLLNGYLADALGRRQTVVVAVVIFCVGVIVQACTKNAGYVFAGRFVTGLGVGNLSMIVPLYNAELAPPEIRGSLVAVQQLSITFGIMVSFWIGYGTNYIGGTGDGQSIAAWEVPVCIQVLPALILAAGMVLFMPQSPRHLMNQGREEECLQTLARLRDAPTDDILVRIEYLEIKSLKMFEEETAKKKYPQYQDGSFKSNFMIGFYDYLSLVTNPSLFKRTTVACLVMLFQQWNGINAINYYAPQVFEGLELGGNTTSLLATGVAGIFEFVFTIPAVLWVDNIGRKKTLLAGAIGMAVCHFICAGLIGSYEGTFGEHKSAGWATVAFVWIFIINFAYSWGPCAWIVVSEVFPLSMRAKGVSIGGSSNWLNNFGVGLATSPFIAASTYGTFIFFGCITVVGAIYVWFFVPETKGRTLEEMDELFGSEGMAAEDEALRQRIDRDIGLTALLHGEMIDSSEKVPEKAVHADDVMLTPPSA

SEQ ID NO: 19 - DNA, 1623 bp, Aspergillus niger
  CDS (1)..(1623)
atggctatcggcaatctttacttcattgcggccatcgccgtcgtcggcggtggtctgttcggtttcgatatctcgtcgatgtcggccatcatcgagaccgatgcctatctctgttacttcaaccaggctcctgtcacttacgatgatgatggcaagagggtctgtcagggccccagcgcgagtgtgcagggtggtatcaccgcctccatggctggtggttcctggttgggctcgttgatctcgggtttcatctcggacaggcttggtcgtcgtactgccattcagatcggttccatcatctggtgcattggatccatcattgtctgtgcctcccagaacattcccatgctgatcgtcggtcgtatcatcaacggtctgagtgtgggtatctgctccgctcaggtgccagtgtatatttcggagattgctcctccaaccaagcgtggtcgtgtcgtcggtctgcaacaatgggctattacctggggtatcctgatcatgttctacgtctcctatggatgcagcttcatcaagggtacggcggccttccggattccctggggtctgcagatgatccctgccgtgctattgttcctgggtatgatgctcctgcctgagtcaccccgctggctggcacgcaaggaccgatgggaggagtgccacgctgttttgaccctcgtccacggtcagggagacccgagctctccctttgtgcagcgtgaatatgaagagatcaagagcatgtgcgagtttgagcgccaaaacgcggatgtctcctacctcgagctgttcaagcccaacatgcttaaccgtacccatgtgggtgttttcgttcagatctggtctcagttgactggaatgaacgtcatgatgtactacatcacctacgtctttgccatggccggcttgaaaggtaacaacaacttgatctcctccagtatccagtacgtgatcaacgtgtgcatgactgtgccggctctggtgtggggtgatcagtggggccgtcgcccgaccttcttgatcggttccctcttcatgatgatctggatgtacattaatgctggtctgatggccagctacggtcatcccgcgccgcccggcggtctcaacaacgtggaagccgagtcctgggtcatccacggcgcgcccagcaaggctgtcattgccagtacctacctcttcgtagcctcatacgccatctccttcggccccgccagctgggtgtacccgccggaactcttccctctgcgtgtgcgcggcaaggctaccgccctctgcacttcagccaactgggccttcaacttcgccctcagctattttgtccccccggcatttgtcaacatccagtggaaggtctacatcctcttcggtgtcttctgtactgccatgttcttgcacattttcttcttctttcccgagaccacgggtaagaccctggaagaggtcgaggccatcttcactgatcccaatggtattccgtacatcggtactcccgcctggaagacaaagaacgagtactcgcgcggtgcacacattgaggaggttggctttgaagatgagaagaaggttgctggtgggcagactatccaccaggaggtcacggctactccggataagattgcttga

SEQ ID NO: 20 - protein, 540 aa, Aspergillus niger
MAIGNLYFIAAIAVVGGGLFGFDISSMSAIIETDAYLCYFNQAPVTYDDDGKRVCQGPSASVQGGITASMAGGSWLGSLISGFISDRLGRRTAIQIGSIIWCIGSIIVCASQNIPMLIVGRIINGLSVGICSAQVPVYISEIAPPTKRGRVVGLQQWAITWGILIMFYVSYGCSFIKGTAAFRIPWGLQMIPAVLLFLGMMLLPESPRWLARKDRWEECHAVLTLVHGQGDPSSPFVQREYEEIKSMCEFERQNADVSYLELFKPNMLNRTHVGVFVQIWSQLTGMNVMMYYITYVFAMAGLKGNNNLISSSIQYVINVCMTVPALVWGDQWGRRPTFLIGSLFMMIWMYINAGLMASYGHPAPPGGLNNVEAESWVIHGAPSKAVIASTYLFVASYAISFGPASWVYPPELFPLRVRGKATALCTSANWAFNFALSYFVPPAFVNIQWKVYILFGVFCTAMFLHIFFFFPETTGKTLEEVEAIFTDPNGIPYIGTPAWKTKNEYSRGAHIEEVGFEDEKKVAGGQTIHQEVTATPDKIA

SEQ ID NO: 21 - DNA, 1542 bp, Aspergillus niger
  CDS (1)..(1542)
atgtatcgcatttcgaatatctacgtcctggctggcttcggcaccattggcggtgccttgttcggcttcgatgtcagctccatgagtgcctggattggcacggatcagtatctggaatacttcaaccacccagattccgacttgcaaggtggtattaccgcttccatgtctgctggctcctttgccggtgcactcgctgccggcttcatatcagaccgaattggtcgtcgttactcccttatgctagcgtgttgcatctgggtcatcggcgctgcgattcaatgtagcgcccaaaatgtcgcccacctggtcgccgggagagttatcagcggcctgtccgtcggtattacatcctcacaggtctgcgtttatctcgctgaactagctccggctcggattcgtggtcgtatcgtcggcattcagcaatgggccatcgaatggggtatgctcatcatgtatctgatctcgtacggatgcggccaaggtctggctggagcagcttctttccgagtatcatggggtgttcagggtatcccggccctcatcctacttgccgcattacccttcttccccgaatctcctcgttggctggcaagtaaagagcgctgggaggaggctttggatactttagcgctgctgcatgccaagggcgaccgcaacgatcccgtggtccaagtggaatacgaagaagtgcaggaagcagcacgcattgcccaggaggccaaggacatttctttcttttcactgttcggaccaaagatttggaagcgaaccctttgcggtgtcagcgcccaggtctggcagcagttgctcggaggcaatgttgcgatgtactacgtcgtgtacatcttcaacatggctggcatgtctggaaacaccaccctgtactcgtcagccatccagtacgtcatcttcttagtcacaacgggtactattctgccatttgttgatcgtatcggtcgacggcttttgctcctcactggatccgtcctctgcatggcttgccactttgctattgctggtcttatggcttcacgtggtcatcatgttgactcggtcgatggcaatgcgaacctgaagtggtcaattacgggaccacccggcaaaggtgtcatcgcctgctcgtacatcttcgttgccgtttatggtttcacctgggccccagtagcatggatctatgcttctgaagtcttcccccttaagtaccgtgcaaagggtgtcggtctctcagctgcgggtaactggattttcaacttcgcgttggcgtactttgtggctcctgctttcaccaacattcagtggaagacttatatcatcttcggtgtcttttgcactgtgatgacgttccatgtgttctttttctacccggagactgctcgacgatcgcttgaagacattgacttgatgtttgagacggacatgaagccctggaagacgcaccagattcatgaccgcttcggcgaagaagttgagcgacacaagcacaaggacatggctgaccaagaaaagggagttgtgtcgacccatgacgaaatggcgtga

SEQ ID NO: 22 - protein, 513 aa, Aspergillus niger
MYRISNIYVLAGFGTIGGALFGFDVSSMSAWIGTDQYLEYFNHPDSDLQGGITASMSAGSFAGALAAGFISDRIGRRYSLMLACCIWVIGAAIQCSAQNVAHLVAGRVISGLSVGITSSQVCVYLAELAPARIRGRIVGIQQWAIEWGMLIMYLISYGCGQGLAGAASFRVSWGVQGIPALILLAALPFFPESPRWLASKERWEEALDTLALLHAKGDRNDPVVQVEYEEVQEAARIAQEAKDISFFSLFGPKIWKRTLCGVSAQVWQQLLGGNVAMYYVVYIFNMAGMSGNTTLYSSAIQYVIFLVTTGTILPFVDRIGRRLLLLTGSVLCMACHFAIAGLMASRGHHVDSVDGNANLKWSITGPPGKGVIACSYIFVAVYGFTWAPVAWIYASEVFPLKYRAKGVGLSAAGNWIFNFALAYFVAPAFTNIQWKTYIIFGVFCTVMTFHVFFFYPETARRSLEDIDLMFETDMKPWKTHQIHDRFGEEVERHKHKDMADQEKGVVSTHDEMA

SEQ ID NO: 23 - DNA, 1512 bp, Aspergillus niger
  CDS (1)..(1512)
atgcgtctctccccagcatggtatcaattcctagtaggcgttttcgcctcgctaggttcattcttatatggatacgatttaggtgtgattgctgaagtcatcgcctgcgggtcgttcatatccagattcaatttgaatgacactgaatctggacttgtggtgtcgatgttcactgcaggtgctttctttggtgcagcgtttgcaggtccaagtggcgacaagctaggcagacgatggaccattactgtcggttgtgttctcttctgcctcggaggtggtcttcagactggagcacggactgtcgcctacctctatagcggaaggttctttgctggactgggggtcggcttccttactatgattatccctctttaccaggcggagatctgccatccggacatacgtggtcgtgttactgctctgcagcaattcatgctaggagttgggtctctatgtgccgcttggatctcatacggcacatatattggattttccgagaccaatgacacacaatggcagctaccccttgggctgcaaattgcaccagctgtgttcctgggacttttgatcatgcttttccctgagtcaccacggtggctcattgatcacggccagcacgagaaaggcttgaagacattggcaatgcttcatgcccacggcaacgaagaggatccttgggttcgtgcggaatttaaccagattcaagaaagcattgtttacgagcatgagaacgaagccaagtcctacaaggaactctttacctcacgatcgtccttccgtcgcctgtttctttgttgctcattacaggcatctgtacagatgaccggcgtgtctgcaatccaatactactctgtaaccatctatggtcagattggcattagtggcgacaaaacgctacagtatcaagctatcaactccatcatcgcccttgtagctcaattcctatgcattctcttcattgaccgagtcggtcgccgctggagtttgatctggggtaacctaggaaacatggtaacgtttattgttgcctgcattctgttagcacagttccccccagaaagccacaacaccggagcccactggggctttatcatcatgacttggctatacaacttctccttctcgtgcacatgtggccctctctcctggatcatcccagcagaggtattcgacacccgaacgcggtcgaagggcgtttcgttggctactatgacatcgtatgcattcaacacgatgattgggcaggtgacgcccattgccatggagagtgtgcgctatcggtactatttcctctttatcatctgcaacttcacgaacgcggtgttcttttggctgttgcttcctgagacgaagaaattgccgcttgaagaaatgaactacctgttctcgaattctccgtggattgtggcgggtaagcggaaggaggattatgttcctcacgatttacagcgcaggcttgaggaggaggcggagaagagagaggtgtttgcgacccacaatgaagtgagttga

SEQ ID NO: 24 - protein, 503 aa, Aspergillus niger
MRLSPAWYQFLVGVFASLGSFLYGYDLGVIAEVIACGSFISRFNLNDTESGLVVSMFTAGAFFGAAFAGPSGDKLGRRWTITVGCVLFCLGGGLQTGARTVAYLYSGRFFAGLGVGFLTMIIPLYQAEICHPDIRGRVTALQQFMLGVGSLCAAWISYGTYIGFSETNDTQWQLPLGLQIAPAVFLGLLIMLFPESPRWLIDHGQHEKGLKTLAMLHAHGNEEDPWVRAEFNQIQESIVYEHENEAKSYKELFTSRSSFRRLFLCCSLQASVQMTGVSAIQYYSVTIYGQIGISGDKTLQYQAINSIIALVAQFLCILFIDRVGRRWSLIWGNLGNMVTFIVACILLAQFPPESHNTGAHWGFIIMTWLYNFSFSCTCGPLSWIIPAEVFDTRTRSKGVSLATMTSYAFNTMIGQVTPIAMESVRYRYYFLFIICNFTNAVFFWLLLPETKKLPLEEMNYLFSNSPWIVAGKRKEDYVPHDLQRRLEEEAEKREVFATHNEVS

SEQ ID NO: 25 - DNA, 1260 bp, Aspergillus niger
  CDS (1)..(1260)
atgtctgagcctaagaaccagccggtagaggagccaaccatcccgcccgaagaaggggtcaagggctggctgacggtggccggaagcttttgcgccctattctcctcctttgggttcctgaatgctatcggagtcttccaaacaacatatcaacagacgtcgctcaaagattacgacgcgtccgacatatcatggattttcgccgtgcagctggccctcatgtgggcgccgggaccgctatggggccgcatgattgacacttatggcccgatcccggtcctctggccatgcagcattctctgtgtgctaggactttgtatgactagcctggcgcacgagtactatcaaatattccttgcgcaaggcctctgcttcggcatcggagccggtggtgttttcacttcggccatgatttgcgtcggccaatggttcgttcgccgccgaggccttgcaactggcattgcggtatcgggaagttcacttgggggtgttatctttcccatattccatgaccgggtcatcaatgacatcggcttctatggtgccgttcggtatacagcgctattcgtcggaattatgctggcaatagcctgcctactgatccgcagccgtcttccgcgcaaagagtggaacggtaaagcagcgtgggtcgacctgactctgctgaaggataccgcatttggattgtacaccgctggcgcatttttcatcatgtggggtctctgggcccctttcgactatatttccagcatggcagagaacgcgggattttcgtcgactcttgctctctacctgatttctatcatcaatgccgcctccatcttcggacgtctcatccctcctcagctagcagacgtgtttggtcacttcaacgtcctgacactctgctgcttcggtactggagtgtcaatgctctgtctttggcttcctttcaactatcacccttcacatgcgggcattatcgtcttctcggcggtctatggcttcgtcagtggcgcggttgtctcccttatgatgccatgtgttgccaaagtgggagatctgcaaaccctgggtcagcgatttggaacatttcagctaatcatgtcagtaagctgcctgaccgggttgccgatcatgggagccatactcgaaaagcaaaattacacggattactcagggttgcagctttttggtgcttgctgtgggatattagggtcagttttgatcggagcagcgacgttcttgctcaggaaaatgcggaatacgagcaaaatatga

SEQ ID NO: 26 - protein, 419 aa, Aspergillus niger
MSEPKNQPVEEPTIPPEEGVKGWLTVAGSFCALFSSFGFLNAIGVFQTTYQQTSLKDYDASDISWIFAVQLALMWAPGPLWGRMIDTYGPIPVLWPCSILCVLGLCMTSLAHEYYQIFLAQGLCFGIGAGGVFTSAMICVGQWFVRRRGLATGIAVSGSSLGGVIFPIFHDRVINDIGFYGAVRYTALFVGIMLAIACLLIRSRLPRKEWNGKAAWVDLTLLKDTAFGLYTAGAFFIMWGLWAPFDYISSMAENAGFSSTLALYLISIINAASIFGRLIPPQLADVFGHFNVLTLCCFGTGVSMLCLWLPFNYHPSHAGIIVFSAVYGFVSGAVVSLMMPCVAKVGDLQTLGQRFGTFQLIMSVSCLTGLPIMGAILEKQNYTDYSGLQLFGACCGILGSVLIGAATFLLRKMRNTSKI

SEQ ID NO: 27 - DNA, 1317 bp, Aspergillus niger
  CDS (1)..(1317)
atggagaacttcgctgatttgtttgcgaaaggtgtcttcctaggaggttatattagcaatacctatggtcggaaatggtgcatatttagcatgaatctctacgcactaggcagtgcagcagtagtggtgagctccaaaacagacgcacaaatattaacaggtcgagcattacactacatctacctaggaatgcaactagccgtaatcccaaccaccctcgccgaactcgccccccggaacgtccgcggcgccatgggcgtcctatactggctcagcatcaaaatcggaggcctagtagtaaccggcatcgcacgaggcacatccggcatctcaacaaacgccgcatggcaaatccccttcggcttgattctcgtgattccatttatttgcatctggctcgtctggataatacccgagtctccgcgatggctgctcctccgcgaccgacaagaagaagcactgcaagcgttgaaacgataccgatcgaaaaccacaccccaacatgagatacaagaagatctcgacgaaatggcgcagaaggtcacggcccagctgcagaagaaaagcttcaaggatctctttatcaagggaaatcgtgaacgtacgttcgttgttgccgctgcgaacttctttcagcaggcttcggggcaggcgtttgcgagtcagtatgggacgcttttcgtcaagcagttgaaatcggtcaatgcgtttagtgttacgctgggtactaatgcgactgatattggggcgttgattatttcgacggcgctgagtgatgtagtgggacggagaatgctcttccacataagcggcttcctcgaaacggcggccttaatgacaatgggcggtctcggtaccgcagacactagtaacactgctgccaaagaaggcatcgtcgctatgcttcttctttacagctttggttggtctcttgcctgggcgccgttggtgtatgtgcttggtgcggagttgccttctgcggggttgagagaaatgactctgcggatcgcgtatactgtgaagctggtgactgagttcgctgtgacattttcgtatccttacctcgaaaccgcagacgatcccggccatgtggacattggtggtaagctgggattcatttatgggtcgctgtctgctgtctcagtcatatttgggtacttcttcatcccagaaacgaggaagttagagttggaggatatcaataaaaagtatgagtcgtcaacggagagtgaaatggagcagaaggttgagtatcagattactctagattctatctatccttgtgcgccgattcctctccgtgtaaatgatggttaa

SEQ ID NO: 28 - protein, 438 aa, Aspergillus niger
MENFADLFAKGVFLGGYISNTYGRKWCIFSMNLYALGSAAVVVSSKTDAQILTGRALHYIYLGMQLAVIPTTLAELAPRNVRGAMGVLYWLSIKIGGLVVTGIARGTSGISTNAAWQIPFGLILVIPFICIWLVWIIPESPRWLLLRDRQEEALQALKRYRSKTTPQHEIQEDLDEMAQKVTAQLQKKSFKDLFIKGNRERTFVVAAANFFQQASGQAFASQYGTLFVKQLKSVNAFSVTLGTNATDIGALIISTALSDVVGRRMLFHISGFLETAALMTMGGLGTADTSNTAAKEGIVAMLLLYSFGWSLAWAPLVYVLGAELPSAGLREMTLRIAYTVKLVTEFAVTFSYPYLETADDPGHVDIGGKLGFIYGSLSAVSVIFGYFFIPETRKLELEDINKKYESSTESEMEQKVEYQITLDSIYPCAPIPLRVNDG

SEQ ID NO: 29 - DNA, 1528 bp, Aspergillus niger
  CDS (1)..(1506)
atgggtcgatactttacaattggcctggctgcatttgcagctacggggtcatatctgttcggatacgacagcggtgttatgacggacgtaatcgagtctaaaaactttctggctttcttcaacaccacccagacatcatccatcatcggtgctatcaacagcacattttcggggggtgcttgcattggtgcgctccagggagggctgacgatggatcgcttcggacgaaagttcactatccaaatgggtgctttcatatgcatgattggcgctattctccagtcgtcagcgaagaatttggctatgattcttgttggtcgcattctggccggctgggctgttggtctcatgtcgatgtctgttccagtgtatcaagcagaggtcgctcatccccgttcgcgtgggttcatcatcggtcttgcccagcagatgatcggtgttggattcattgtcagtacctgggtcggctacggctcccttcatgcaccgaataccagcgagttccaatggcgattcccgcttgcctttcaggcagtccctgcagttctcctggttataggcatgtttttcatgcccgagtcacctcggtacctgatcgaaaaggaacgctacgaggaagccatgaaaatcctccggagacttcacttcgacggcaccaacgaggactggattcagactgagtacaacgagatcaaaaccaccattgaagccgaaaaagctgtcacagttcccggctggctcatcatgtttcgtgtgccccaatggcgaacccgactcatgcacgggatcgccgtccaagtattcacccagatgacaggcgtcaacgtcgtgaactactatcaaacaatcatgtacaatgccctcggcatcactggtaaccgcaacaccctcgtagcaggaatctacaactgcgtcggtcccatcacgaacttcattttcatctttttccttctcgaccgagttgggcgccgcaagcccatgttgttcggaacaatcgctatcaccattgccctggtctgcgaagcagctctctactcgcagaacctggacggcactcgaaagggatacagtatcggtggtgttttcttcatcttctgcatcactgttatcttttctctgtcatttggtccttgcagttgggtctacatggcagaagtaatgccaatgcaaatccgcggtcgaggaaacgccttcgccactgcaatcggcaactgggccgtaagcactctctggaaccaggtctccccgatcgcgctggacaaaattcagtggaagttttatttcgtgtttgcggcgtggaacgtatgcatcaccctcccaaccgtctacttcttctttaaagagacaaatcaaaaatcactcgaagagatcgatctactctttggtggtcgtgccctgggaatgcttcctgaggatgtggctgccaagggaggaacagtggagactgagaaggcagaggagacgggggtatcggttattaatgttgagcatacgagtactgtgtgaccatgacggagtgtgtgaattc

SEQ ID NO: 30 - protein, 501 aa, Aspergillus niger
MGRYFTIGLAAFAATGSYLFGYDSGVMTDVIESKNFLAFFNTTQTSSIIGAINSTFSGGACIGALQGGLTMDRFGRKFTIQMGAFICMIGAILQSSAKNLAMILVGRILAGWAVGLMSMSVPVYQAEVAHPRSRGFIIGLAQQMIGVGFIVSTWVGYGSLHAPNTSEFQWRFPLAFQAVPAVLLVIGMFFMPESPRYLIEKERYEEAMKILRRLHFDGTNEDWIQTEYNEIKTTIEAEKAVTVPGWLIMFRVPQWRTRLMHGIAVQVFTQMTGVNVVNYYQTIMYNALGITGNRNTLVAGIYNCVGPITNFIFIFFLLDRVGRRKPMLFGTIAITIALVCEAALYSQNLDGTRKGYSIGGVFFIFCITVIFSLSFGPCSWVYMAEVMPMQIRGRGNAFATAIGNWAVSTLWNQVSPIALDKIQWKFYFVFAAWNVCITLPTVYFFFKETNQKSLEEIDLLFGGRALGMLPEDVAAKGGTVETEKAEETGVSVINVEHTSTV

SEQ ID NO: 31 - DNA, 1545 bp, Aspergillus niger
  CDS (1)..(1545)
atgagattaccaaaaattaaaatgcctcgtaagaggccgcaagatgaactcaatggtgatgtgctgcagcaagaggcgcaagctaccggccccaatggccagaaatctgaagaggggattgtggatacacctatccctctgctcacgtggcgttctttcttaatgggcattttcgtgtcaatgggtggtttcctcttcggctacgacactggccaaatttcaggcttcttggagatgccgaactttctccaaagatacggacaacagcaagctgacggaacatactatttctccaatgcccgttcgggtctgattgtcgcgctgctttcaataggtaccttgatcggcgcccttattgcagctcccatcgcagatcgtgttggccggaaatggtccatcagcggctggagtgccatggtgtgtgtcggtatcaccatacaaatatcttcccctttcggaaagtggtaccaggtagccatgggtcgttgggtcgctggtctcggcgttggggctctatcattgcttgttcccatgtaccaagcggaaactggaccgaggcacattcgtggatctcttgttagcacttatcagcttttcatcacccttggaatcttcgtcgccaactgtatcaacttcggaacagaagctcgaaatgatacaggctcctggcgtattcccatgggcatcacctacatctgggccatgatcctgggcttcggaattgcactattccccgaaagtgcccggtacgactatagacatggaagggaggcgaaagcagctcgtaccctatcgagaataagttcgtggaaggaacaggaacgcggagagataacctggtctcatcttttccatgcaccccgcatgaagtatcgcgtcgcagtgggagtcgcccttcaagcactgcaacagttaacgggagcgaattactttttctactacgggacgacgatctttcgaggcgccgggatttcaaattcctacgtaacacaaatgattcttggcggtgtgaatttcggaacgacgttcctagggctgtacctgatcgagaactacggtcgtcgtcgatccctgataactggagcattgtggatgtttgtttgtttcatggttttcgcctccgtgggccatttctctctagaccatgaaaacccagaacggacacacacagctggcgtggtcatggttgttttcgcctgtcttttcattctcggtttcgcatcaacatggggtcccatggtgtggaccattatcgcagagctgtatccctccgaattccgtgcccgcgccatgtctctcgccacagcatccaattggctctggaacttccttctcgcctttttcacccccttcatcaccagtgccattgacttccgcctcggctacgtctttgccggctgcctcttccttgcggcgggcctggtttacgtggcagtcattgaaggtcgtggccgtacattggaagagatcgataccatgtacgttatgaaggtgccgccctggaaagagctccaagtatgtatttccggacatcgatccatatga

SEQ ID NO: 32 - protein, 514 aa, Aspergillus niger
MRLPKIKMPRKRPQDELNGDVLQQEAQATGPNGQKSEEGIVDTPIPLLTWRSFLMGIFVSMGGFLFGYDTGQISGFLEMPNFLQRYGQQQADGTYYFSNARSGLIVALLSIGTLIGALIAAPIADRVGRKWSISGWSAMVCVGITIQISSPFGKWYQVAMGRWVAGLGVGALSLLVPMYQAETGPRHIRGSLVSTYQLFITLGIFVANCINFGTEARNDTGSWRIPMGITYIWAMILGFGIALFPESARYDYRHGREAKAARTLSRISSWKEQERGEITWSHLFHAPRMKYRVAVGVALQALQQLTGANYFFYYGTTIFRGAGISNSYVTQMILGGVNFGTTFLGLYLIENYGRRRSLITGALWMFVCFMVFASVGHFSLDHENPERTHTAGVVMVVFACLFILGFASTWGPMVWTIIAELYPSEFRARAMSLATASNWLWNFLLAFFTPFITSAIDFRLGYVFAGCLFLAAGLVYVAVIEGRGRTLEEIDTMYVMKVPPWKELQVCISGHRSI